(19) United States
(12) Patent Application Publication
(10) Pub. No.: US 2022/0008309 A1
(43) Pub. Date: Jan. 13, 2022

(54) COSMETIC PRODUCT FOR TREATING A KERATIN MATERIAL, COMPRISING A FILM-FORMING AND/OR SETTING CONSTITUENT

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); TORSTEN LECHNER, Langenfeld (DE)

(73) Assignee: Henkel AG & Co. KGaA, Duesseldorf (DE)

(21) Appl. No.: 17/289,578

(22) PCT Filed: Oct. 31, 2019
(86) PCT No.: PCT/EP2019/079764

§ 371 Date: Apr. 28, 2021

(30) Foreign Application Priority Data
Oct. 31, 2018 (DE) 10 2018 218 654.5

(51) Int. Cl. A61K 8/58; A61K 8/81; A61Q 5/00; A61Q 5/06

(52) U.S. Cl. CPC A61K 8/585; A61K 8/8117; A61K 2800/591; A61Q 5/06; A61Q 5/004

(57) ABSTRACT

The application discloses a cosmetic agent for treating a keratinous material comprising a) at least one organic silicon compound and b) polystyrene sulfonate(s), where the cosmetic composition that includes these components improves the fastness properties, in particular UV stability, perspiration fastness and/or wash fastness, of dyed keratinous materials, in particular keratinous fibers.

### CROSS-REFERENCE TO RELATED APPLICATION

This application is a U.S. National-Stage entry under 35 U.S.C. § 371 based on International Application No. PCT/EP2019/079764, filed Oct. 31, 2019, which was published under PCT Article 21(2) and which claims priority to German Application No. 10 2018 218 654.5, filed Oct. 31, 2018, which are all hereby incorporated in their entirety by reference.

### TECHNICAL FIELD

The application concerns cosmetic agents for the treatment of a keratinous material with a film-forming and/or setting ingredient, and their use.

### BACKGROUND

Temporary styling for a longer period up to several days usually requires the application of strengthening agents. Corresponding agents for temporary forming usually contain synthetic polymers and/or waxes as the strengthening active ingredient. Agents for the temporary reshaping of keratinous fibers can be made up, for example, as hair spray, hair wax, hair gel, or hair foam.

The most important property of an agent for temporary shaping of hair, hereinafter also referred to as a styling agent, is to give the treated fibers the strongest possible hold in the newly modeled shape—i.e., a shape imprinted on the hair. One also speaks of strong hairstyle hold or the high hold of the styling agent. The hold of the hairstyle is essentially determined by the type and quantity of the strengthening active ingredient used, although the other components of the styling agent may also have an influence.

In addition to a high degree of hold, styling products must meet a whole range of other requirements. These can be roughly divided into properties on the hair, properties of the respective formulation, e.g., properties of sprayed aerosol, and properties relating to the handling of the styling product, with the properties on the hair being of particular importance. Particularly noteworthy are moisture resistance, low tack, and a balanced conditioning effect. Furthermore, a styling product should, if possible, be universally applicable to all hair types and be mild to hair and skin.

To meet the different requirements, a variety of synthetic polymers have already been developed as setting agents for use in styling products.

WO 2018/115059 A1 describes organosilicon compounds from the group of silanes comprising at least one hydroxy group and/or hydrolysable group. Due to the presence of the hydroxy groups and/or hydrolysable groups, the silanes are reactive substances that hydrolyze or oligomerize or polymerize in the presence of water. The oligomerization or polymerization of the silanes, initiated by the presence of the water, ultimately leads to the formation of a film when applied to a keratinous material.

Not every polymer or polymer blend is fundamentally suitable to produce hair styling products.

Although suitable polymers and polymer combinations for use in the field of temporary hair shaping have been under development for some time, the results obtained so far still leave room for improvement, particularly about the storage stability, applicability, and degree of hold of these agents. Currently available styling agents can still be improved in that a good combination of degree of hold and long-term hold (high humidity curl retention) is not always sufficiently guaranteed.

### BRIEF SUMMARY

Cosmetic compositions for treating keratinous material and methods of treating keratinous material are provided. In an exemplary embodiment, a cosmetic composition for treating keratinous material includes at least one organic silicon compound and a polystyrene sulphonate.

A method for treating keratinous material is provided in another embodiments. The method includes applying a cosmetic composition to the keratinous material, where the cosmetic composition includes at least one organic silicon compound and a polystyrene sulphonate.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One task of the present disclosure was to provide further suitable active ingredient combinations which are exemplified by good film-forming and/or strengthening properties and have an extremely high degree of retention without having to sacrifice flexibility and good moisture resistance—especially resistance to perspiration and water.

This task is solved by a cosmetic agent for the treatment of a keratinous material, comprising

a) at least one organic silicon compound and
b) Polystyrene sulfonate(s).

The combination of the two components a) and b) leads to a strongly over-additive, i.e., synergistic, effect about the moisture resistance and the degree of hold of the agent. Surprisingly, the combination of the two components a) and b) has also been shown to improve the fastness properties, in particular UV stability, perspiration fastness and/or wash fastness, of dyed keratinic materials, especially keratinic fibers.

By a keratinous material is meant hair, the skin, the nails (such as fingernails and/or toenails). Wool, furs, and feathers also fall under the definition of keratinous material.

Preferably, a keratinous material is understood to mean human hair, human skin, and human nails, in particular fingernails and toenails. Very preferably, keratinous material is understood to mean human hair, in particular head and/or beard hair.

As a first ingredient essential to the present disclosure, the composition for treating a keratinous material comprises at least one organic silicon compound.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolysable groups per molecule.

In a most preferred embodiment, the agent for treating a keratinous material comprises at least one organic silicon compound selected from silanes having one, two or three silicon atoms, wherein the organic silicon compound further comprises one or more basic groups and one or more hydroxyl groups or hydrolysable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆)alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R′R″R″′Si—O—CH₂—CH₃. The residues R′, R″ and R″′ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),

where
R₁, R₂ independently represent a hydrogen atom or a C₁-C₆ alkyl group,
L is a linear or branched C₁-C₂₀ divalent alkylene group,
R₃ represents a hydrogen atom or a C₁-C₆ alkyl group,
R₄ represents a C₁-C₆ alkyl group
a, stands for an integer from 1 to 3, and
b stands for the integer 3-a,

(R₅O)_{c}(R₆)_{d}Si-(A)_{c}-[NR₇-(A′)]_{f}-[O-(A″)]_{g}-[NR₈-(A″′)]ₕ-Si(R₆′)_{d′}(OR_{5′})_{c′} (II),

where
R5, R5′, R5″ independently represent a hydrogen atom or a C₁-C₆ alkyl group,
R6, R6′ and R6″ independently represent a C₁-C₆ alkyl group,
A, A′, A″, A″′ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R_{5′}, R_{5″}, R₆, R_{6′}, R_{6″}, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, where preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, and the 3-aminopropyl group. The 2-aminoethyl group is particularly preferred. Examples of a linear divalent C₁-C₂₀ alkylene group include the methylene group (—CH₂)), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

Preferably, -L- represents a linear, divalent C₁-C₂₀ alkylene group. Further preferably, -L- represents a linear divalent C₁-C₆ alkylene group. Particularly preferred -L-stands for a methylene group (CH₂—), an ethylene group (—CH₂—CH₂—), a propylene group (—CH₂—CH₂—CH₂—) or a butylene (—CH₂—CH₂—CH₂—CH₂—). L very particularly stands for a propylene group (—CH₂—CH₂—CH₂—).

The organic silicon compounds of formula (I)

R₁R₂N-L-Si(OR₃)ₐ(R₄)_{b} (I),

each have the silicon-containing group —Si(OR₃)ₐ(R₄)_{b} at one end.

In the terminal structural unit —Si(OR₃)ₐ(R₄)_{b}, R₃ is hydrogen or C₁-C₆ alkyl group, and R₄ is C₁-C₆ alkyl group. R₃ and R₄ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The strongest improvement in terms of moisture resistance and degree of retention, as well as fastness when applied to colored keratinous materials, was obtained when the agent for treating a keratinous material contains at least one organic silicon compound of the formula (I) in which the radicals R₃ and R₄, independently of one another, represent a methyl group or an ethyl group.

Furthermore, the strongest improvement in terms of moisture resistance and degree of retention as well as fastness when applied to colored keratinous materials was obtained when the agent for treating a keratinous material contains at least one organic silicon compound of the formula (I) in which the radical a represents the number 3. In this case the remainder b stands for the number 0.

In another preferred embodiment, an agent for treating a keratinous material is exemplified in that it comprises at least one organic silicon compound of formula (I), where
R₃, R₄ independently of one another represent a methyl group or an ethyl group and
a stands for the number 3 and
b stands for the number 0.

In another preferred embodiment, the composition for treating a keratinous material comprises at least one organic silicon compound of formula (I)

R1R₂N-L-Si(OR₃)ₐ(R₄)_{b} (I),

where
R₁, R₂ both represent a hydrogen atom,
L represents a linear, divalent C₁-C₆-alkylene group, preferably a propylene group (—CH₂—CH₂—CH₂—) or an ethylene group (—CH₂—CH₂—),
R₃ represents a hydrogen atom, an ethyl group, or a methyl group,
R₄ represents a methyl group or an ethyl group,
a stands for the number 3, and
b stands for the number 0.

Particularly well-suited organic silicon compounds of formula (I) are
(3-Aminopropyl)triethoxysilane

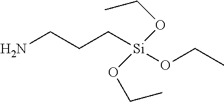

(3-Aminopropyl)trimethoxysilane

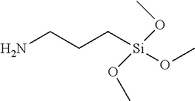

1-(3-Aminopropyl)silanetriol

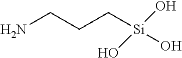

(2-Aminoethyl)triethoxysilane

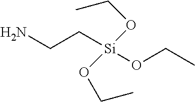

(2-Aminoethyl)trimethoxysilane

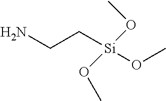

1-(2-Aminoethyl)silanetriol

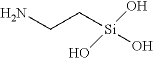

(3-Dimethylaminopropyl)triethoxysilane

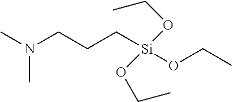 [SMILES: CCO[Si](CCCN(C)C)(OCC)OCC]

(3-Dimethylaminopropyl)trimethoxysilane

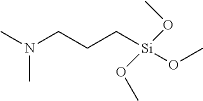

1-(3-Dimethylaminopropyl)silanetriol

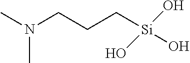

(2-Dimethylaminoethyl)triethoxysilane,

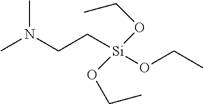

(2-Dimethylaminoethyl)trimethoxysilane and/or

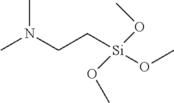

1-(2-Dimethylaminoethyl)silanetriol

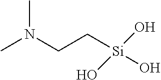

The organic silicon compound of formula (I) is commercially available. (3-aminopropyl)trimethoxysilane, for example, can be purchased from Sigma-Aldrich®. (3-Aminopropyl)triethoxysilane is also commercially available from Sigma-Aldrich®.

In another embodiment, the composition for treating a keratinous material comprises at least one organic silicon compound of formula (II)

(R₅O)_{c}(R₆)_{d}Si-(A)_{c}-[NR₇-(A′)]_{f}-[O-(A″)]_{g}-[NR₈-(A″′)]ₕ-Si(R₆′)_{d′}(OR₅′)_{c′} (II).

The organosilicon compounds of formula (II) each bear at their two ends the silicon-containing groupings (R₅O)_{c}(R₆)_{d}Si— and —Si(R₆′)_{d′}(OR₅′)_{c′}.

In the central part of the molecule of formula (II) there are the groups -(A)_{c}- and —[NR₇-(A′)]_{f}-

and —[O-(A″)]_{g}- and —[NR₈-(A″′)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group including -(A)- and —[NR₇-(A′)]- and —[O-(A″)]- and —[NR₈-(A″′)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here c stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

An extremely high anti-pollution effect of the agent for the treatment of a keratinous material could be obtained when the residues c and c′ both stand for the number 3. In this case d and d′ both stand for the number 0.

In another preferred one, the agent for treating a keratinous material comprises at least one organic silicon compound of formula (II)

(R₅O)_{c}(R₆)_{d}Si-(A)ₑ-[NR₇-(A′)]_{f}-[O-(A″)]_{g}-[NR₈-(A″′)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

When c and c′ both represent the number 3 and d and d′ both represent the number 0, the organic silicon compounds correspond to formula (IIa)

(R₅O)₃Si-(A)ₑ-[NR₇-(A′)]_{f}-[O-(A″)]_{g}-[NR₈-(A″′)]ₕ-Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)e- and -[NR7-(A′)]f- and -[O-(A″)]g- and -[NR8-(A″′)]h- are in the middle part of the organic silicon compound of formula (II).

In this context, the presence of certain groupings has proven to be particularly beneficial in terms of increasing the “anti-pollution” effect. Particularly good results were obtained when at least two of the residues e, f, g, and h stand for the number 1. Especially preferred e and f both stand for the number 1. Furthermore, g and h both stand for the number 0.

When e and f are both 1 and g and h are both 0, the organic silicon compounds are represented by the formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A″′ and A″″ independently represent a linear or branched divalent C₁-C₂₀ alkylene group. Preferably the radicals A, A′, A″, A″′ and A″″ independently of one another represent a linear, divalent C₁-C₂₀ alkylene group. Further preferably the radicals A, A′, A″, A″′ and A″″ independently represent a linear divalent C₁-C₆ alkylene group. In particular, the radicals A, A′, A″, A″′ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A″′ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NR₈-(A″)]. If the radical R7 now stands for a grouping of the formula (III), the agent for treating a keratinous material contains an organic silicon compound with 3 reactive silane groups.

In another preferred one, the agent for treating a keratinous material comprises at least one organic silicon compound of formula (II)

(R₅O)_{c}(R₆)_{d}Si-(A)ₑ-[NR7-(A′)]_{f}-[O-(A″)]_{g}-[NR₈-(A″′)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In another preferred embodiment, the composition for treating a keratinous material comprises at least one organic silicon compound of the formula (II), wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are
3-(trimethoxysilyl)-N-[3-(trimethoxysilyl)propyl]-1-propanamine

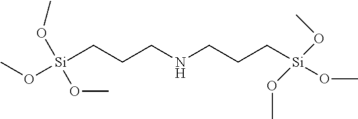

3-(Triethoxysilyl)-N-[3-(triethoxysilyl)propyl]-1-propanamine

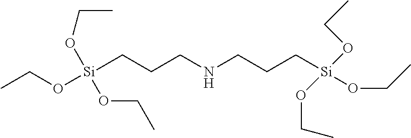 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

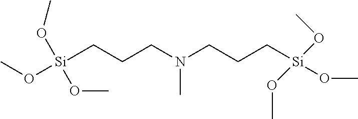

N-Methyl-3-(triethoxysilyl)-N-[3-(triethoxysilyl)propyl]-1-propanamine

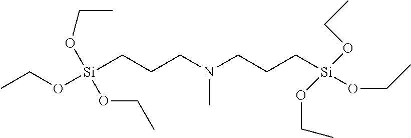 [SMILES: CCO[Si](CCCN(C)CCC[Si](OCC)(OCC)OCC)(OCC)OCC]

2-[Bis[3-(trimethoxysilyl)propyl]amino]-ethanol

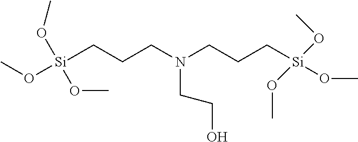

2-[bis[3-(triethoxysilyl)propyl]amino]ethanol

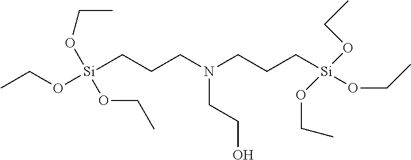 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

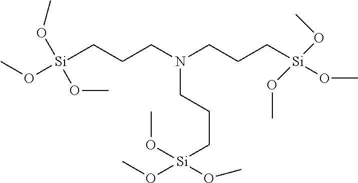

3-(Triethoxysilyl)-N,N-bis[3-(triethoxysilyl)propyl]-1-propanamine

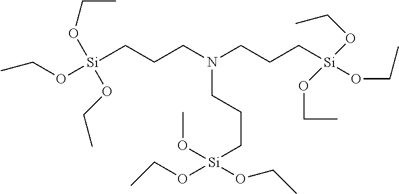 [SMILES: CCO[Si](CCCN(CCC[Si](OCC)(OCC)OCC)CCC[Si](OCC)(OCC)OCC)(OC)OCC]

N1,N1-Bis[13-(trimethoxysilyl)propyl]-1,2-ethanediamine,

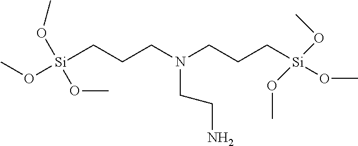

N1,N1-Bis[3-(triethoxysilyl)propyl]-1,2-ethanediamine,

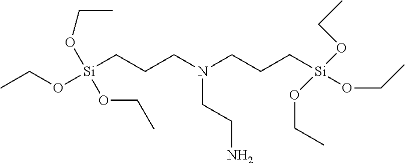 [SMILES: CCO[Si](CCCN(CCN)CCC[Si](OCC)(OCC)OCC)(OCC)OCC]

N,N-Bis[3-(trimethoxysilyl)propyl]-2-propen-1-amine

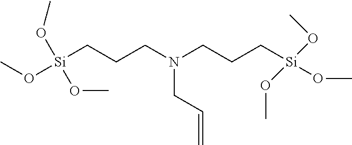

N,N-Bis[3-(triethoxysilyl)propyl]-2-propen-1-amine

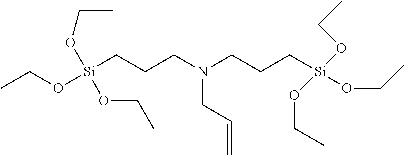 [SMILES: C=CCN(CCC[Si](OCC)(OCC)OCC)CCC[Si](OCC)(OCC)OCC]

The organic silicon compounds of formula (II) are commercially available. Bis(trimethoxysilylpropyl)amine with the CAS number 82985-35-1 can be purchased from Sigma-Aldrich®.

Bis[3-(triethoxysilyl)propyl]amine with the CAS-Number 13497-18-2 can be purchased from Sigma-Aldrich®, for example.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich® or Fluorochem®.

3-(triethoxysilyl)-N,N-bis[3-(triethoxysilyl)propyl]-1-propanamine with the CAS number 18784-74-2 can be purchased for example from Fluorochem® or Sigma-Aldrich®.

It has also been found to be particularly advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer 3-k.

In a further preferred embodiment, the composition for treating a keratinous material contains, in addition to the organic silicon compound or compounds of formula (I), at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a likewise preferred embodiment, the composition for treating a keratinous material contains, in addition to the organic silicon compound or compounds of the formula (II), at least one further organic silicon compound of the formula (IV) contains

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In another preferred embodiment, the composition for treating a keratinous material contains, in addition to the organic silicon compounds of the formula (I) and (II), at least one further organic silicon compound of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₅ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. Particularly preferably, R10 represents a methyl group or an ethyl group.

In the organic silicon compounds of formula (IV), the radical R₁₁ represents a C₁-C₅ alkyl group. Particularly preferably, R₁₁ represents a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

A strong improvement in terms of moisture resistance and degree of retention, as well as fastness when applied to colored keratinous materials, could be obtained when the agent for treating a keratinous material contains at least one organic silicon compound of formula (IV) in which the radical k represents the number 3. In this case the residue m stands for the number 0.

Organic silicon compounds of the formula (IV) that are particularly suitable for solving the problem are
Methyltrimethoxysilane

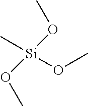

Methyltriethoxysilane

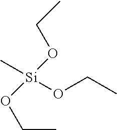

Ethyltrimethoxysilane

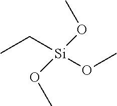

Ethyltriethoxysilane

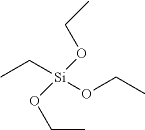

n-Hexyltrimethoxysilane

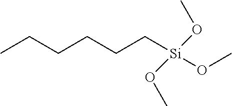

n-Hexyltriethoxysilane

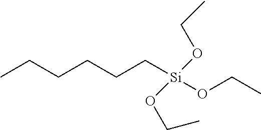 [SMILES: CCCCCC[Si](OCC)(OCC)OCC]

n-Octyltrimethoxysilane

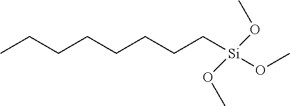

n-Octyltriethoxysilane

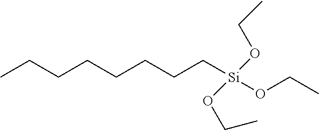 [SMILES: CCCCCCCC[Si](OCC)(OCC)OCC]

n-dodecyltrimethoxysilane and/or

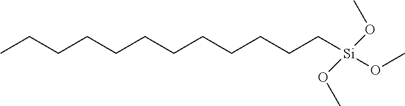 [SMILES: CCCCCCCCCCCC[Si](OC)(OC)OC]

n-dodecyltriethoxysilane.

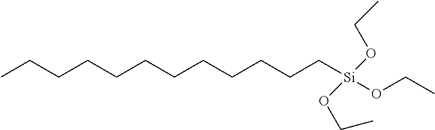 [SMILES: CCCCCCCCCCCC[Si](OCC)(OCC)OCC]

and propyltrimethoxysilane, propyltriethoxysilane, octadecyltrimethoxysilane and/or octadecyltriethoxysilane.

The organic silicon compounds described above are reactive compounds. In this context, it has been found to be preferred if the agent for treating a keratinous material contains—based on the total weight of the agent for treating a keratinous material—one or more organic silicon compounds in a total amount of from about 0.1 to about 30% by weight, preferably from about 0.2 to about 25% by weight, particularly preferably from about 0.25 to about 20% by weight and most preferably from about 0.5 to about 15% by weight.

To achieve a particularly good improvement in terms of moisture resistance and degree of retention as well as fastness when used with colored keratinous materials, it is particularly advantageous to use the organic silicon compounds of formula (I) and/or (II) in certain ranges of amounts in the agent for treating a keratinous material. The agent for treating a keratinous material contains—based on the total weight of the agent for treating a keratinous material—one or more organic silicon compounds of the formula (I) and/or (II) in a total amount of from about 0.1 to about 10% by weight, preferably from about 0.2 to about 5% by weight and particularly preferably from about 0.5 to about 3% by weight is particularly preferred.

Furthermore, it has been found to be particularly preferred if the organic silicon compounds of formula (IV) are also contained in the agent for treating a keratinous material in certain ranges of amounts. The agent for treating a keratinous material contains—based on the total weight of the agent for treating a keratinous material—one or more organic silicon compounds of the formula (IV) in a total amount of from about 0.1 to about 20% by weight, preferably from about 2 to about 15% by weight and particularly preferably from about 4 to about 9% by weight.

In the course of the work leading to the present disclosure, it was found that particularly stable and uniform films could be obtained on the keratinous material if the agent for treating a keratinous material contained at least two organic silicon compounds that were structurally different from one another.

In another preferred embodiment, an agent for treating a keratinous material contains two structurally different organic silicon compounds.

In an explicitly very particularly preferred embodiment, the composition for treating a keratinous material comprises at least one organic silicone compound of formula (I) selected from the group including (3-aminopropyl)triethoxysilane and (3-aminopropyl)trimethoxysilane, and additionally contains at least one organic silicone compound of formula (IV) selected from the group including methyltrimethoxysilane, methyltriethoxysilane, ethyltrimethoxysilane and ethyltriethoxysilane.

In another preferred embodiment, the composition for treating a keratinous material comprises at least one organic silicone compound of formula (II) selected from the group including (bis(trimethoxysilylpropyl)amine, bis[3-(trimethoxysilyl)propyl]amine and N-methyl-3-(trimethoxysilyl)-N-[3-(trimethoxysilyl)propyl]-1-propanamine, and additionally contains at least one organic silicone compound of formula (IV) selected from the group including methyltrimethoxysilane, methyltriethoxysilane, ethyltrimethoxysilane and ethyltriethoxysilane.

In another preferred embodiment, an agent for treating a keratinous material is exemplified in that it comprises, based on the total weight of the cosmetic agent:
from about 0.5 to about 3% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane, (2-dimethylaminoethyl)trimethoxysilane, and (2-dimethylaminoethyl)triethoxysilane, and
from about 3.2 to about 10% by weight of at least one second organic silicon compound selected from the group including methyltrimethoxysilane, methyltriethoxysilane, ethyltrimethoxysilane, ethyltriethoxysilane, propyltrimethoxysilane, propyltriethoxysilane, octyltrimethoxysilane, octyltriethoxysilane, dodecyltrimethoxysilane, dodecyltriethoxysilane, octyldecyltrimethoxysilane and octyldecyltriethoxysilane.

Even the addition of small amounts of water leads to hydrolysis in organic silicon compounds with at least one hydrolysable group. The hydrolysis products and/or organic silicon compounds having at least one hydroxy group may react with each other in a condensation reaction. For this reason, both the organosilicon compounds having at least one hydrolysable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolysable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

A second essential ingredient of the agent for treating a keratinous material is polystyrene sulfonate(s).

Preferred compositions are exemplified in that they contain from about 0.1 to about 10% by weight, preferably from about 0.5 to about 8% by weight and more preferably from about 1 to about 6% by weight, in each case based on the total weight of the composition for treating a keratinous material, of polystyrene sulfonate(s).

The polystyrene sulfonate can be modified by copolymerization with suitable comonomers, such as acrylonitrile or butadiene, or by blending with other polymers.

Quite preferred agents are exemplified in that they contain sodium polystyrene sulfonate(s) having structural units of the formula (PS)

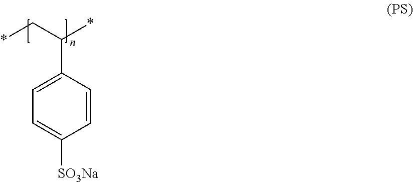

in which n represents values from about 100 to about 1500, preferably from about 200 to about 1250, more preferably from about 300 to about 1000 and from about 400 to about 800.

Preferred compositions are exemplified in that they contain from about 0.1 to about 10% by weight, preferably from about 0.5 to about 8% by weight and more preferably from about 1 to about 6% by weight, in each case based on the total weight of the composition for treating a keratinous material, sodium polystyrene sulfonate

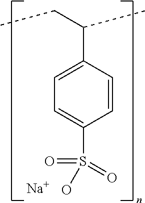

with n=from about 100 to about 1000, preferably from about 200 to about 900, more preferably from about 300 to about 800, still more preferably from about 400 to about 750 and about 600 to about 700.

Preferred combinations of active ingredients include:

(3-aminopropyl)trimethoxysilane and sodium polystyrene sulfonate,
(3-aminopropyl)triethoxysilane and sodium polystyrene sulfonate,
(2-aminoethyl)trimethoxysilane and sodium polystyrene sulfonate,
(2-aminoethyl)triethoxysilane and sodium polystyrene sulfonate,
(3-dimethylaminopropyl)trimethoxysilane and sodium polystyrene sulfonate,
(3-dimethylaminopropyl)triethoxysilane and sodium polystyrene sulfonate,
(2-dimethylaminoethyl)trimethoxysilane and sodium polystyrene sulfonate,
(2-dimethylaminoethyl)triethoxysilane and sodium polystyrene sulfonate,
3-(trimethoxysilyl)-N-[3-(trimethoxysilyl)propyl]-1-propanamine and sodium polystyrene sulfonate,
3-(triethoxysilyl)-N-[3-(triethoxysilyl)propyl]-1-propanamine and sodium polystyrene sulfonate,
N-methyl-3-(trimethoxysilyl)-N-[3-(trimethoxysilyl)propyl]-1-propanamine and sodium polystyrene sulfonate,
N-methyl-3-(triethoxysilyl)-N-[3-(triethoxysilyl)propyl]-1-propanamine and sodium polystyrene sulfonate,
2-[bis[3-(trimethoxysilyl)propyl]amino]ethanol and sodium polystyrene sulfonate,
2-[bis[3-(triethoxysilyl)propyl]amino]ethanol and sodium polystyrene sulfonate,
3-(trimethoxysilyl)-N,N-bis[3-(trimethoxysilyl)propyl]-1-propanamine and sodium polystyrene sulfonate,
3-(trimethoxysilyl)-N,N-bis[3-(trimethoxysilyl)propyl]-1-propanamine and sodium polystyrene sulfonate,
N1,N1-bis[3-(trimethoxysilyl)propyl]-1,2-ethanediamine and sodium polystyrene sulfonate,
N1,N1-bis[3-(triethoxysilyl)propyl]-1,2-ethanediamine and sodium polystyrene sulfonate,
N,N-bis[3-(trimethoxysilyl)propyl]-2-propen-1-amine and sodium polystyrene sulfonate,
N,N-bis[3-(triethoxysilyl)propyl]-2-propen-1-amine and sodium polystyrene sulfonate,
Methyl trimethoxysilane and sodium polystyrene sulfonate,
Methyltriethoxysilane and sodium polystyrene sulfonate,
Ethyl trimethoxysilane and sodium polystyrene sulfonate,
Ethyltriethoxysilane and sodium polystyrene sulfonate,
Octyltrimethoxysilane and sodium polystyrene sulfonate,
Octyltriethoxysilane and sodium polystyrene sulfonate,
Dodecyl trimethoxysilane and sodium polystyrene sulfonate,
Dodecyltriethoxysilane and sodium polystyrene sulfonate,
(3-aminopropyl)trimethoxysilane, methyltrimethoxysilane and sodium polystyrene sulfonate,
(3-aminopropyl)triethoxysilane, methyltrimethoxysilane and sodium polystyrene sulfonate,
(3-aminopropyl)triethoxysilane, methyltriethoxysilane and sodium polystyrene sulfonate,
(3-aminopropyl)triethoxysilane, ethyltrimethoxysilane and sodium polystyrene sulfonate,
(3-aminopropyl)triethoxysilane, ethyltriethoxysilane and sodium polystyrene sulfonate,
(3-aminopropyl)triethoxysilane, propyltrimethoxysilane and sodium polystyrene sulfonate,
(3-aminopropyl)triethoxysilane, propyltriethoxysilane and sodium polystyrene sulfonate,
(3-aminopropyl)triethoxysilane, hexyltrimethoxysilane and sodium polystyrene sulfonate,
(3-aminopropyl)triethoxysilane, hexyltriethoxysilane and sodium polystyrene sulfonate,
(3-aminopropyl)triethoxysilane, octyltrimethoxysilane and sodium polystyrene sulfonate,
(3-aminopropyl)triethoxysilane, octyltriethoxysilane and sodium polystyrene sulfonate,
(3-aminopropyl)triethoxysilane, dodecyltrimethoxysilane and sodium polystyrene sulfonate,
(3-aminopropyl)triethoxysilane, dodecyltriethoxysilane and sodium polystyrenesulfonate,
(3-aminopropyl)triethoxysilane, octadecyltrimethoxysilane and sodium polystyrenesulfonate,
(3-aminopropyl)triethoxysilane, octadecyltriethoxysilane and sodium polystyrene sulfonate,
3-(trimethoxysilyl)-N-[3-(trimethoxysilyl)propyl]-1-propanamine, methyl trimethoxysilane and sodium polystyrenesulfonate,
3-(triethoxysilyl)-N-[3-(triethoxysilyl)propyl]-1-propanamine, methyl trimethoxysilane and sodium polystyrenesulfonate,
N-methyl-3-(trimethoxysilyl)-N-[3-(trimethoxysilyl)propyl]-1-propanamine, methyl trimethoxysilane and sodium polystyrenesulfonate,
N-methyl-3-(triethoxysilyl)-N-[3-(triethoxysilyl)propyl]-1-propanamine, methyl trimethoxysilane and sodium polystyrenesulfonate,
(3-aminopropyl)trimethoxysilane, methyltriethoxysilane and sodium polystyrene sulfonate,
(3-aminopropyl)triethoxysilane, methyltriethoxysilane and sodium polystyrene sulfonate,
3-(Trimethoxysilyl)-N-[3-(trimethoxysilyl)propyl]-1-propanamine, methyltriethoxysilane and sodium polystyrenesulfonate,
3-(triethoxysilyl)-N-[3-(triethoxysilyl)propyl]-1-propanamine, methyltriethoxysilane and sodium polystyrenesulfonate,
N-methyl-3-(trimethoxysilyl)-N-[3-(trimethoxysilyl)propyl]-1-propanamine, methyltriethoxysilane and sodium polystyrenesulfonate,
N-methyl-3-(triethoxysilyl)-N-[3-(triethoxysilyl)propyl]-1-propanamine, methyltriethoxysilane and sodium polystyrenesulfonate.

In particular, the composition for treating a keratinous material may comprise components for temporarily reshaping a keratinous material.

In the following, further ingredients of the agent for the treatment of a keratinous material are described, which may be included in the agents in addition to the previously described mandatory ingredients.

It may be preferred that the agent for treating a keratinous material further comprises a setting compound, preferably selected from the group including waxes, synthetic polymers, and mixtures thereof.

To meet the different requirements for agents for the treatment of a keratinous material in the form of an agent for the temporary reshaping of a keratinous material (=styling agent), many synthetic polymers have already been developed as strengthening compounds, which can be used in the agent for the treatment of a keratinous material. Alternatively, or complementarily, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic, and amphoteric strengthening polymers.

Suitable synthetic polymers include, for example, polymers with the following INCI designations: Acrylamides/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Ammonium Methacrylate Copolymer, Acrylates/t-Butyl Acrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetone Acrylamide Copolymer, Ammonium-Acrylates/VA/Acrylates Copolymer, AMPD-Acrylates/Acrylamides Copolymer, AMPD-Acrylates/Acrylamides/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Acrylate Crosspolymer, Lauryl Methacrylate/Glycol Dimethacrylate Crosspolymer, MEA-Sulphite, Methacrylic Acid/Sodium Acrylamidomethyl Propane Sulphonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophen-2, Polyquaternium-2 Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polquaternium-56 9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a setting compound.

Preferably, the setting compound comprises a vinylpyrrolidone-containing polymer. Particularly preferably, the setting compound comprises a polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel®.

Accordingly, it is particularly preferred that the setting compound comprises a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

The cosmetic composition for treating a keratinous material may contain, in addition or as an alternative to a synthetic polymer, at least one natural or synthetic wax having a melting point above 37° C. as a setting compound.

Natural or synthetic waxes can be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate, furthermore synthetic full esters of fatty acids and glycols (for example Syncrowax®) or polyols with 2 to 6 C atoms, fatty acid monoalkanolamides with a C12-22 acyl radical and a C2-4 alkanol radical, esters of saturated and/or unsaturated, branched and/or unbranched alkanecarboxylic acids with a chain length of 1 to 80 C atoms and saturated and/or unsaturated branched and/or unbranched alcohols of a chain length of 1 to 80 C atoms, including, for example, synthetic fatty acid fatty alcohol esters such as stearyl stearate or cetyl palmitate, esters of aromatic carboxylic acids, dicarboxylic acids or hydroxycarboxylic acids (for example, 12-hydroxystearic acid) and saturated and/or unsaturated branched and/or unbranched alcohols having a chain length of 1 to 80 carbon atoms, lactides of long-chain hydroxycarboxylic acids and full esters of fatty alcohols and di- and tricarboxylic acids, for example dicetyl succinate or dicetyl/stearyl adipate, and mixtures of these substances.

The wax components can also be selected from the group of esters of saturated, unbranched alkanecarboxylic acids having a chain length of 14 to 44 carbon atoms and saturated, unbranched alcohols having a chain length of 14 to 44 carbon atoms, provided that the wax component or the totality of wax components are solid at room temperature. The wax components can be selected, for example, from the group of C16-36 alkyl stearates, C10-40 alkyl stearates, C2-40 alkyl isostearates, C20-40 dialkyl esters of dimer acids, C18-38 alkyl hydroxystearoyl stearates, C20-40 alkyl erucates, and C30-50 alkyl beeswax and cetearyl behenate. Silicone waxes, for example stearyltrimethylsilane/stearyl alcohol, may also be beneficial. Preferred wax components are the esters of saturated monohydric C20-C60 alcohols and saturated C8-C30 monocarboxylic acids, in particular a C20-C40 alkyl stearate preferably available under the name Kesterwachs® K82H from Koster Keunen® Inc.

Natural, chemically modified, and synthetic waxes can be used alone or in combination. Thus, several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl® & Co), Polywax® GP 200 (a mixture of stearyl alcohol and polyethylene glycol stearate with a melting point of about 47-51° C.; manufacturer: Croda®) and “Softceresin® FL 400” (a vaseline/vaseline oil/wax mixture with a melting point of about 50-54° C.; manufacturer: Parafluid Mineral Oil™ Company) are examples of mixtures that can be used.

Preferably, the wax is selected from carnauba wax (INCI: Copernicia Cerifera Cera) Bienenwachs (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and petrolatum.

The wax or wax components should be solid at about 25° C. and should melt in the range of >about 37° C.

The composition for treating a keratinous material preferably contains the setting compound in a total amount of from about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include surfactants, cationic polymers, protein hydrolysates, amino acids, oligopetides, vitamins, provitamins, vitamin precursors, betaines, bioquinones, purine (derivatives), taurine (derivatives), conditioning agents, plant extracts, polydimethylsiloxanes, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

The active ingredient combination of at least one organic silicon compound and at least one polystyrene sulfonate may already be present in the composition for treating a keratinous material. In this embodiment, the agent for treating a keratinous material is already distributed in a form ready for use. To provide a formulation that is as stable as possible during storage, the agent itself is preferably packaged with low or no water.

Alternatively, the at least one organic silicon compound, optionally together with one or more further ingredients of the composition for treating a keratinous material, is added to a base comprising all ingredients of the composition for treating a keratinous material, with the exception of the at least one organic silicon compound, a maximum of about 12 hours, preferably a maximum of about 6 hours, more preferably a maximum of about 1 hour, still more preferably a maximum of about 30 minutes, and very particularly preferably a maximum of about 20 minutes, before application of the composition for treating a keratinous material.

For example, the user may first mix or shake an agent (α) comprising the organic silicon compound(s) and optionally other ingredients of the agent for treating a keratinous material with an agent (β) comprising the remaining ingredients of the agent for treating a keratinous material. The user can now apply this mixture of (α) and (β)—either directly after its preparation or after a short reaction time of from about 10 seconds to about 20 minutes—to the keratinous materials. The agent (β) may contain water, in particular water in an amount of >about 30% by weight, based on the total weight of the agent for treating keratinous materials.

A further subject of the present application is the use of a cosmetic agent as contemplated herein for treating a keratinous material to improve the fastness properties, in particular UV stability, perspiration fastness and/or wash fastness, of dyed keratinous materials, dyed keratinous fibers. The dyed keratinous fibers are especially preferably dyed human hair.

About further preferred embodiments of use, the same applies mutatis mutandis as to the cosmetic agents.

The cosmetic agent as contemplated herein for treating a keratinous material can also be used for temporary deformation of human hair, preferably dyed human hair.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for treating a keratinous material comprising:
a) at least one organic silicon compound; and
b) a polystyrene sulfonate(s).

2. The cosmetic composition for treating a keratinous material according to claim 1, wherein said at least one organic silicon compound comprises a silane having one, two or three silicon atoms, said organic silicon compound comprising one or more hydroxyl groups or hydrolysable groups per molecule.

3. The cosmetic composition for treating a keratinous material according to claim 1, wherein the at least one organic silicon compound comprises a compound of formula (I) and/or (II), in which the organic silicon compound of formula (I) is
R₁R₂N-L-Si(OR₃)ₐ(R₄)_{b} (I),
R₁, R₂ independently represent a hydrogen atom or a C₁-C₆ alkyl group,
L is a linear or branched C₁-C₂₀ divalent alkylene group,
R₃, R₄ independently of one another represent a C₁-C₆ alkyl group,
a, stands for an integer from 1 to 3, and
b stands for the integer 3-a, and
wherein in the organic silicon compound of formula (II) is
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR_{5′})_{c′} (I),
R₅, R_{5′}, R_{5′″}, R₆, R_{6′} and R_{6′″} independently represent a C₁-C₆ alkyl group,
A, A′, A″, A″′ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d″}(OR₅″)_{c″} (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the residues e, f, g, and h is different from 0.

4. The cosmetic composition for treating a keratinous material according to claim 1, wherein the composition for treating a keratinous material comprises at least one organic silicon compound of formula (I),
R₁R₂N-L-Si(OR₃)ₐ(R₄)_{b} (I),
where
R₁, R₂ both represent a hydrogen atom,
L represents a linear, divalent C₁-C₆-alkylene group,
R₃, R₄ independently represent a methyl group or an ethyl group,
a stands for the number 3 and
b stands for the number 0.

5. The cosmetic composition for treating a keratinous material according to claim 1, wherein the composition for treating a keratinous material comprises at least one organic silicon compound of formula (I) selected from the group of:
(3-aminopropyl)trimethoxysilane
(3-aminopropyl)triethoxysilane
(2-aminoethyl)trimethoxysilane
(2-aminoethyl)triethoxysilane
(3-dimethylaminopropyl)trimethoxysilane
(3-dimethylaminopropyl)triethoxysilane
(2-dimethylaminoethyl)trimethoxysilane and
(2-dimethylaminoethyl)triethoxysilane.

6. The cosmetic composition for treating a keratinous material according to claim 1, wherein the composition for treating a keratinous material comprises at least one organic silicon compound of formula (II) selected from the group of:
3-(trimethoxysilyl)-N-[3-(trimethoxysilyl)propyl]-1-propanamine
3-(triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine, and
N,N-bis[3-(triethoxysilyl)propyl]-2-propen-1-amine.

7. The cosmetic composition for treating a keratinous material according to claim 1, wherein the composition for treating a keratinous material comprises at least one organic silicon compound of formula (IV),
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

8. The cosmetic composition for treating a keratinous material according to claim 1, wherein the composition for treating a keratinous material comprises at least one organic silicon compound of formula (IV),
R₉Si(OR₁₀)ₖ(R₁₁)ₘ (IV),
which is selected from the group of:
methyltrimethoxysilane
methyltriethoxysilane
ethoxysilane
ethyltriethoxysilane
propyltrimethoxysilane
propyltriethoxysilane
hexyltrimethoxysilane
hexyltriethoxysilane
octyltrimethoxysilane
octyltriethoxysilane
dodecyltrimethoxysilane
dodecyltriethoxysilane
octadecyltrimethoxysilane and
octadecyltriethoxysilane.

9. The cosmetic composition for treating a keratinous material according to claim 1, wherein the composition for treating a keratinous material comprises at least two structurally different organic silicon compounds.

10. A method of treating keratinous material comprising the steps of: applying the cosmetic composition to the keratinous material, where the cosmetic composition comprises an organic silicon compound and a polystyrene sulfonate.

11. The cosmetic composition according to claim 1, wherein the cosmetic composition comprises the at least one organic silicon compound in an amount of from about 0.1 to about 30% by weight, based on a total weight of the cosmetic composition.

12. The cosmetic composition according to claim 11, wherein the cosmetic composition comprises the polystyrene sulfonate(s) in an amount of from about 0.1 to about 10% by weight, based on the total weight of the cosmetic composition.

13. The cosmetic composition according to claim 3, wherein the cosmetic composition comprises the at least one organic silicon compound of the formula (I) and/or the formula (II) in an amount of from about 0.2 to about 5% by weight, based on a total weight of the cosmetic composition.

14. The cosmetic composition according to claim 7, wherein the cosmetic composition comprises the at least one organic silicon compound of the formula (IV) in an amount of from about 2 to about 15% by weight, based on a total weight of the cosmetic composition.

15. The cosmetic composition according to claim 9, wherein the cosmetic composition comprises at least one organic silicone compound selected from the group of:
(3-aminopropyl)triethoxysilane, and
(3-aminopropyl)trimethoxysilane, and additionally comprises at least one organic silicone compound selected from the group of:
methyltrimethoxysilane,
methyltriethoxysilane,
ethyltrimethoxysilane, and
ethyltriethoxysilane.

16. The cosmetic composition according to claim 9, wherein the cosmetic composition comprises from about 0.5 to about 3% by weight of at least one first organic silicon compound selected from the group of:
(3-aminopropyl)trimethoxysilane,
(3-aminopropyl)triethoxysilane,
(2-aminoethyl)trimethoxysilane,
(2-aminoethyl)triethoxysilane,
(3-dimethylaminopropyl)trimethoxysilane,
(3-dimethylaminopropyl)triethoxysilane,
(2-dimethylaminoethyl)trimethoxysilane, and
(2-dimethylaminoethyl)triethoxysilane, and from about 3.2 to about 10% by weight of at least one second organic silicon compound selected from the group of:
methyltrimethoxysilane,
methyltriethoxysilane,
ethyltrimethoxysilane,
ethyltriethoxysilane,
propyltrimethoxysilane,
propyltriethoxysilane,
octyltrimethoxysilane,
octyltriethoxysilane,
dodecyltrimethoxysilane,
dodecyltriethoxysilane,
octyldecyltrimethoxysilane and
octyldecyltriethoxysilane, wherein the weight percents are base on a total weight of the cosmetic composition.

17. The cosmetic composition according to claim 11, wherein the cosmetic composition comprises the polystyrene sulfonate(s) in an amount of from about 1 to about 10% by weight, based on the total weight of the cosmetic composition.

18. The cosmetic composition according to claim 1, wherein the cosmetic composition further comprises a setting compound selected from the group of a wax, a synthetic polymer, and combinations thereof.

19. The cosmetic composition according to claim 18, wherein the cosmetic composition comprises the setting compound in an amount of from about 0.5 to about 50% by weight, based on a total weight of the cosmetic composition.

20. The method of treating keratinous material of claim 10, further comprising: adding the at least one organic silicon compound to the cosmetic composition a maximum of about 12 hours before applying the cosmetic composition to the hair.
